(19) United States
(12) Patent Application Publication
(10) Pub. No.: US 2024/0139451 A1
(43) Pub. Date: May 2, 2024

(54) CONNECTORS AND ASSEMBLIES

(71) Applicant: SMITHS MEDICAL INTERNATIONAL LIMITED, Ashford (GB)

(72) Inventors: Usamah Hanif, Southend-on-Sea (GB); Steven Mark Tupper, Hythe (GB)

(73) Assignee: SMITHS MEDICAL INTERNATIONAL LIMITED, Ashford (GB)

(21) Appl. No.: 17/769,356

(22) PCT Filed: Oct. 13, 2020
(86) PCT No.: PCT/GB2020/000086

§ 371 Date: Apr. 15, 2022

(30) Foreign Application Priority Data
Oct. 22, 2019 (GB) 1915251.1

(51) Int. Cl. A61M 16/04

(52) U.S. Cl. CPC A61M 16/0463

(57) ABSTRACT

A closed system suction catheter (3) is connected with a tracheal tube (1) by means of a Y-shape connector (2) having one port (202) connected with the tracheal tube, a second port (203) connected with a breathing tube (200) and a third port (204) connected with the closed system suction catheter. The third port of the connector includes a normally-closed flap valve (320) that is opened by insertion of the patient end fitting (306) of the closed system suction catheter and closes when the suction catheter is removed from the connector so that ventilation pressure is maintained and contamination is prevented.

This invention relates to connectors of the kind for fitting with a tracheal tube, breathing tubing and a suction device, the connector having a first port adapted to fit with the tracheal tube, a second port opening into the first port and adapted to fit with an end of the breathing tubing and a third port adapted to fit with the suction device such that the suction device can be advanced through the third and first ports into the tracheal tube.

The invention is also concerned with assemblies of tracheal tubes, connectors and suction catheters assemblies.

Suction catheter assemblies may be used to remove secretions from within a tracheal tube or the respiratory passages of a patient. They are also used in other applications. Suction catheter assemblies may be of the closed-system kind in which the catheter is enclosed within a flexible envelope. Such assemblies have a manifold at the patient end with a sliding seal through which a suction catheter can be advanced and withdrawn. The flexible envelope is joined at one end to the manifold and encloses the catheter along its length. The other end of the envelope and the catheter are joined with a rear, machine end housing including a suction control valve and a connector. The connector connects the catheter to a suction source and the valve enables the clinician to control the suction applied by the catheter.

Examples of closed-system suction catheter assemblies are described in U.S. Pat. Nos. 5,269,768, 5,300,043, 4,569,344, 4,638,539, 4,872,579, 5,167,622, 5,779,687, 5,325,850, 5,490,503, 5,419,769, 5,460,613, 5,349,950, GB2394761, GB2400160, U.S. Pat. Nos. 6,109,259, 6,227,197, EP801577B, WO96/09082, EP1239907B, EP1478424B, U.S. Pat. No. 6,588,427, EP1620148B, US2004/0221852, EP1911482A, EP1795217A, US2007/0282250, WO2007/143502, US2008/0188833, U.S. Pat. Nos. 6,227,200, 6,543,451, EP1239909B, U.S. Pat. No. 6,602,219, EP1347798, WO02/49680, U.S. Pat. No. 6,609,520, WO/055143, U.S. Pat. Nos. 6,805,125, 6,923,184, 7,021,313, 7,191,782, WO2004/101045, U.S. Pat. No. 7,263,997, WO2004/103448, WO00/15276, EP637257B, EP1113835B, EP1210957A, EP1237612B, U.S. Pat. No. 7,152,603, EP1267957B, U.S. Pat. No. 6,978,783, US2004/0007236, US2005/0211253, US2005/0211245, US2005/0235987, U.S. Pat. No. 7,059,322, WO2004/032817, US2008/0135051, U.S. Pat. Nos. 4,836,199, 4,850,350, 4,967,743, 5,025,806, 5,083,561, 5,220,916, 5,215,522, 5,255,676, 5,277,177, 5,309,902, 5,333,606, 5,343,857, 5,487,381, 5,513,628, 5,791,337, EP1343552A, WO02/49699, U.S. Pat. No. 6,612,304, EP1322371A, WO02/28463, U.S. Pat. No. 6,629,530, WO02/051485, U.S. Pat. No. 6,769,430, EP1330284, WO02/36191, U.S. Pat. No. 6,886,561, WO2004/034946, U.S. Pat. No. 7,188,623, WO2006/014431, U.S. Pat. No. 7,341,059, WO2005/094925, WO2006/103233, WO2007/030388, WO2009/003135, U.S. Pat. Nos. 4,838,255, 5,107,829, 5,133,345, 5,642,726, 6,702,789, 7,458,955, 7,273,473, 5,139,018, 4,327,723, 4,515,592, 6,099,519, EP695556B, U.S. Pat. Nos. 5,065,754, 5,730,123, 5,207,220, 5,309,903, 7,086,402, 7,597,686, 7,726,315, WO11020985, GB2468946, GB1914578.8, GB1911561.7, GB1910834.9, GB1906050.8, GB1902867.9, GB1902868.7, GB1818954.8, GB1816890.6, GB1914426, GB1910630.1, GB1910629.3 and GB1906183.7. Closed system suction catheters are available from various manufacturers including Smiths Medical, Kimberley Clark, Covidien and Viasys.

Where a patient is being ventilated using a tracheostomy tube the ventilator must be continuously connected to the tracheostomy tube via a breathing or ventilation tube. Every few hours the tracheostomy tube must be cleaned using a suction device. In order to avoid the need to disconnect the breathing tube it is common practice to use a Y connector having one limb fitted to the tracheostomy tube connector, a second limb connected to the ventilator via the breathing tube and a third limb connected to the suction device. The suction device has to remain connected to avoid gas venting to atmosphere through the disconnected third limb. Having the suction device continuously connected to the tracheostomy tube in this way, however, creates a problem in that it adds leverage to the machine end of the tracheostomy tube, which can cause discomfort to the patient. Also, it clutters the site of the tracheostomy and can become tangled in tubing or cables in this region. Where the patient is a child the attached suction catheter can create an additional problem in that it makes it difficult for a parent to hold the child, thereby preventing the child benefitting from the physical contact with its parent.

Another problem arises because the suction catheter assembly has to be removed and replaced every few days. Disconnecting a conventional suction catheter assembly from a tracheal tube causes an immediate, although temporary loss of ventilation pressure. This allows the lungs to deflate and can result in the loss of the therapeutic benefit achieved by previous ventilation. A further problem arises when the suction catheter assembly is disconnected because the tracheal tube is opened to atmosphere. This allows contamination to enter the patient and can also result in contamination from the patient venting to atmosphere and being a potential hazard to nearby clinical workers from this contamination, which may be of bacterial, viral or other biological, toxic or radioactive form.

It is an object of the present invention to provide an alternative connector and assembly.

According to one aspect of the present invention there is provided a connector of the above-specified kind, characterised in that the third port includes a valve that is opened by a part of the suction device and closes when the part of the suction device is removed.

The part of the suction device may be a patient end fitting of the suction device such that the patient end fitting engages and opens the valve. The valve may be a duckbill valve or flap valve. The valve may be a tricuspid valve with three leaves of generally triangular shape urged sealingly together in its natural state.

According to another aspect of the present invention there is provided an assembly of a tracheal tube and a connector according to the above one aspect of the present invention, wherein the first port of the connector is fitted with a connector at the machine end of the tracheal tube.

According to a further aspect of the present invention there is provided an assembly of a suction device and a connector according to the above one aspect of the present invention, wherein the third port of the connector is fitted with a fitting at the patient end of the suction device.

The suction device is preferably a closed system suction catheter assembly.

According to a fourth aspect of the present invention there is provided an assembly of a tracheal tube, breathing tubing, a closed system suction catheter assembly and a connector, the connector having a first port connected with the tracheal tube, a second port connected with the breathing tubing, and a third port connected with the closed system suction catheter assembly such that a suction catheter in the closed system suction catheter assembly can be advanced through the third and first ports into the tracheal tube, characterised in that the third port includes a valve that is opened by a part of the closed system suction catheter assembly and closes when the part of the closed system suction catheter assembly is removed.

An assembly of a tracheostomy tube, breathing tube, connector and closed-system suction catheter according to the present invention will now be described, by way of example, with reference to the accompanying drawings, in which:

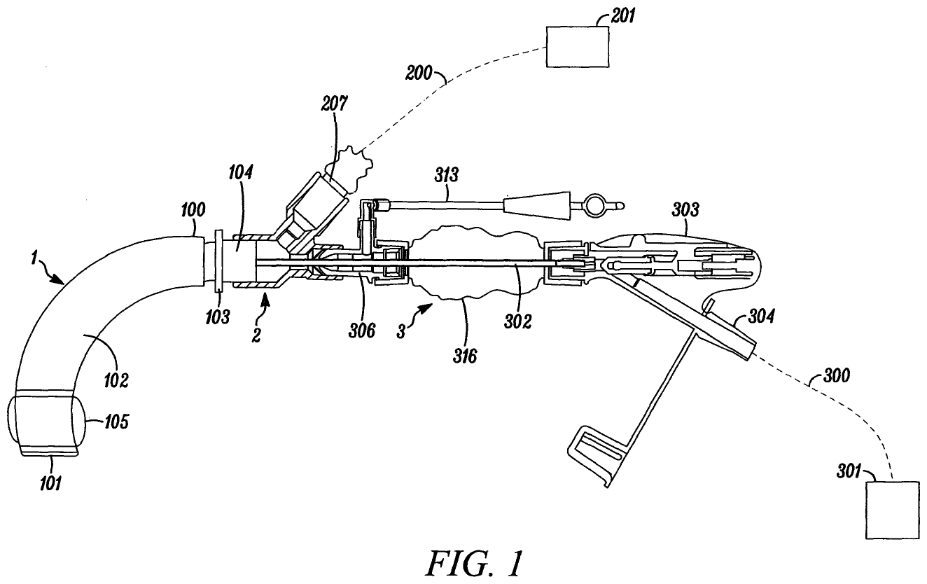

FIG. 1 is a sectional side elevation view of the assembly;

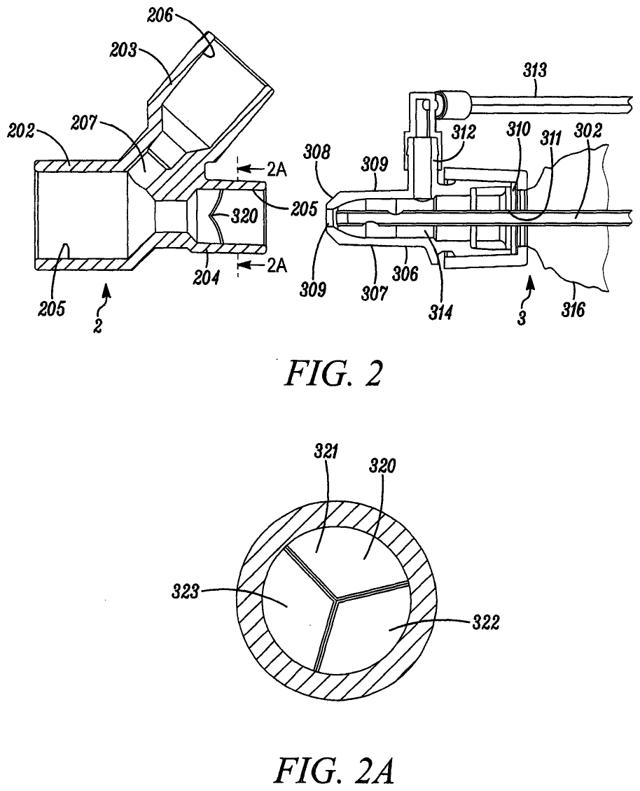

FIG. 2 is a sectional side elevation view of the connector and the patient end of the suction catheter before being fitted together;

FIG. 2A is sectional end view across the suction port of the connector along the line 2A-2A;

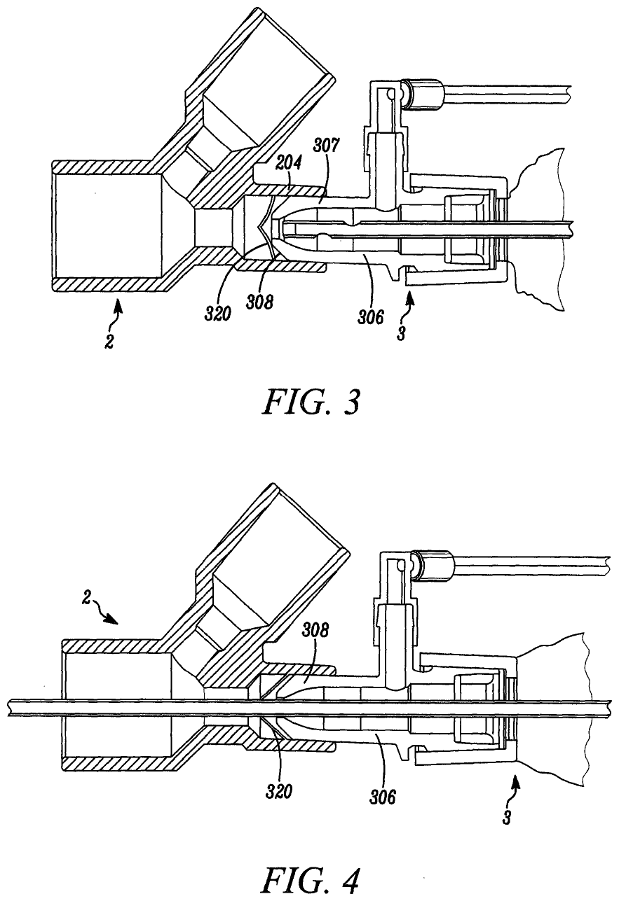

FIG. 3 is a sectional side elevation view of the connector and the patient end of the suction catheter at a preliminary stage of being fitted together;

FIG. 4 is a sectional side elevation view of the connector and patient end of the suction catheter fully fitted together and with the suction catheter extended; and

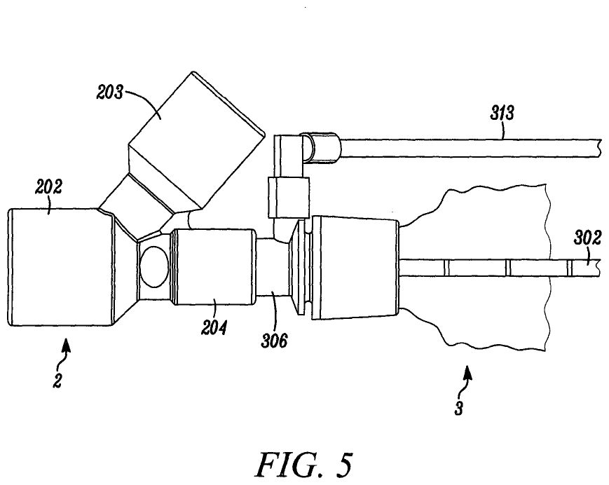

FIG. 5 is a side elevation view of the connector and suction catheter fitted together.

With reference first to FIGS. 1 and 2, the assembly comprises a tracheostomy tube 1 connected at its machine end 100 to one port of a connector 2. A second port of the connector 2 is connected via breathing or ventilation tubing 200 to a ventilator 201. A third port of the connector 2 is connected to the patient end of a suction device in the form of a closed system suction catheter assembly 3, which in turn is connected via suction tubing 300 to a suction source 301.

The tracheostomy tube 1 is entirely conventional having a patient end 101 adapted for location in the trachea, a curved shaft 102 and a neck flange 103 at the machine end 100 of the tube adapted to lie close to the patient's neck surface. A tapered 15 mm male connector 104 is fitted on the machine end 100 of the tube 1. The tube 1 is shown as having an inflatable sealing cuff 105 towards its patient end 101 but the tube could be uncuffed.

The suction catheter assembly 3 is of the closed system kind having a small bore flexible suction catheter 302 attached at its rear, machine end to a suction control valve 303. The suction control valve 303 has a suction inlet 304 connected by the suction tubing 300 to the suction source 301, such as including a pump and collection reservoir. The opposite, patient end of the suction catheter 302 extends into a patient end fitting 306 of the assembly having a nose portion 307 at its patient end with a steeply tapered tip 308 providing a central opening 309 slightly wider than the external diameter of the suction catheter 302. To the rear of this tip 308 the patient end fitting 306 has a shallower external tapered coupling portion 309. At its rear, machine end the patient end fitting 306 houses a wiper seal 310 with a central aperture 311 through which the suction catheter 302 extends in a slidable manner. An irrigation port 312 projects radially outwardly of the patient end fitting 306 midway along its length. The outer end of the irrigation port 312 is coupled to a short irrigation line 313 by which liquid can be introduced into a lavage cavity 314 provided by the interior of the patient end fitting. The suction catheter 302 can be washed by introducing irrigating liquid via the irrigation port 312 into the lavage cavity 314 while suction is applied to the suction catheter, in the usual way. The suction catheter assembly 3 is completed by a flexible sleeve or envelope 316 extending around the outside of the suction catheter 302 and connected at its rear end to the forward end of the suction control valve 303 and at its forward end to the rear of the patient end fitting 306.

The connector 2 is shown most clearly in FIGS. 2 to 5 and differs from previous Y-shape connectors used to connect the machine end of a tracheostomy tube with a ventilator and a suction catheter assembly by the inclusion of a valve 320 that closes when the suction catheter assembly is removed from the connector. The connector 2 is moulded from a clear plastics material as a unitary, one-piece construction with three ports or limbs 202, 203 and 204. The first port 202 at the forward or patient end of the connector 2 is open at its outer end and has a circular section with a tapering bore 205 shaped to provide a female mating coupling that fits on the outside of the male connector 104 on the tracheostomy tube 1. The second port or limb 203 extends generally rearwardly and is inclined relative to the first port 202 at an angle of about 120° and is slightly smaller in external diameter than the first port. The second port 203 is also open at its outer end and has a tapered bore 206 that provides a female coupling to be fitted on the outside of a male connector 207 at the forward or patient end of the ventilation tubing 200. The bore 206 of the second, ventilation port 203 opens unrestricted, via a reduced diameter passage 207 into the bore 205 of the first, tube port 202 so that air or gas can flow freely between these two ports in both directions.

The third port or limb 204 has a circular section and is smaller in diameter than the first port 202, being about two thirds its external diameter. This third, suction port 204 extends rearwardly and is axially aligned with the first port 202. The third port 204 has a tapered bore 205 adapted to make a mating, sealing fit with the outside of the coupling portion 309 of the nose 307 of the patient end fitting 306 of the closed system suction catheter 3. The third port 204 includes an internal valve 320 arranged to be opened by the closed system suction catheter 3 and to close when the catheter is removed. The valve 320 may take various different forms that will allow passage of the suction catheter 302. Typically the valve could be of a flap or duckbill type, such as a tricuspid valve 320 shown in FIG. 2A with three leaves 321, 322 and 323 of generally triangular shape that are urged sealingly together in its natural state. The drawings show the valve 320 as being formed integrally as a single piece with the connector 2 but it could be formed as a separate component that is subsequently fitted inside the suction port 204.

The dimensions of the third, suction port 204 are selected so that, when the nose 307 of the patient end fitting 306 on the suction catheter assembly 3 is sealing fitted in the suction port the tip 308 of the patient end fitting engages the valve 320 sufficiently to open it. FIG. 2 shows the suction catheter assembly 3 before connection to the connector 2. FIG. 3 shows the patient end fitting 306 before full insertion and FIG. 4 shows the same fitting when fully inserted, the leaves 321 to 323 of the valve 320 being opened by engagement with the tip 308 of the fitting. Alternatively, the suction port of the connector could be longer so that the tip of the patient end fitting, when coupled in the suction port, is spaced rearwardly of the valve and does not open it. Instead the valve would be arranged to be opened only when the suction catheter was extended. This might require the valve to be softer and more flexible to ensure that it can be deflected by the suction catheter. This arrangement would have the advantage that, if the suction catheter assembly 3 were left connected to the connector 2 when the suction catheter was retracted back into the assembly and irrigation liquid was supplied to the patient end fitting, the valve in the connector would provide additional security to prevent irrigation liquid flowing into the patient's respiratory passages.

The valve 320 in the connector 2 enables the suction device 3 to be removed when not needed since the valve prevents escape of ventilation gases from the suction port 204. In this way the connector 2 can remain connected to the ventilator 201 at all times since there is no need to disconnect this in order to provide suction. The valved connector assembly enables the suction catheter assembly to be removed and replaced without causing any drop in ventilation pressure applied to the patient. The connector assembly also avoids the problem of possible contamination to or from the patient caused during removal and replacement of the suction catheter assembly. Because the connector assembly enables the suction catheter assembly to be removed without loss of ventilation pressure it enables the patient to be disconnected from the suction catheter assembly except when suction is needed. This avoids discomfort to the patient caused by the weight of the attached suction catheter assembly and also enables child patients to be handled more freely by their parents.

In order to prevent any leakage of ventilation gas through the valved suction port when the suction device is removed the connector could be provided with a cap (not shown) that can be fitted on the suction port of the connector.

The connector is not limited to use with closed system suction catheters but could be used with conventional suction catheters. With such catheters it might be necessary to fit a guide into the suction port so that when the suction catheter is inserted it is directed centrally of the valve.

The invention is not limited to tracheostomy tubes but could be used with other tubes requiring suctioning.

1-8. (canceled)

9. A connector for fitting with a tracheal tube, breathing tubing and a suction device, the connector having a first port adapted to fit with the tracheal tube, a second port opening into the first port and adapted to fit with an end of the breathing tubing and a third port adapted to fit with the suction device such that the suction device can be advanced through the third and first ports into the tracheal tube, characterised in that the third port includes a valve that is opened by a part of the suction device and closes when the part of the suction device is removed.

10. The connector according to claim 1, characterised in that the part of the suction device is a patient end fitting of the suction device such that the patient end fitting engages and opens the valve.

11. The connector according to claim 1, characterised in that the valve is a duckbill valve or a flap valve.

12. The connector according to claim 11, characterised in that the valve is a tricuspid valve with three leaves of generally triangular shape urged sealingly together in its natural state.

13. An assembly of the tracheal tube and the connector according to claim 1, wherein the first port of the connector is fitted with a machine end connector of the tracheal tube.

14. An assembly of the suction device and the connector according to claim 1, wherein the third port of the connector is fitted with a fitting at the patient end of the suction device.

15. The assembly according to claim 14, wherein the suction device is a closed system suction catheter assembly.

16. An assembly of a tracheal tube, breathing tubing, a closed system suction catheter assembly and a connector, the connector having a first port connected with the tracheal tube, a second port connected with the breathing tubing, and a third port connected with the closed system suction catheter assembly such that a suction catheter in the closed system suction catheter assembly can be advanced through the third and first ports into the tracheal tube, characterised in that the third port includes a valve that is opened by a part of the closed system suction catheter assembly and closes when the part of the closed system suction catheter assembly is removed.
